(19) United States
(12) Patent Application Publication
(10) Pub. No.: US 2025/0319304 A1
(43) Pub. Date: Oct. 16, 2025

(54) IMPLANTABLE APPARATUS HAVING HELIX FIXATION WITH VARYING CROSS-SECTION

(71) Applicant: MEDTRONIC, INC., Minneapolis, MN (US)

(72) Inventors: Michael P. Campbell, Blaine, MN (US); Thomas A. Anderson, New Hope, MN (US); Richard Francis, Pine Springs, MN (US); Andrew J. Ries, Lino Lakes, MN (US)

(21) Appl. No.: 18/867,662

(22) PCT Filed: May 25, 2023
(86) PCT No.: PCT/US2023/023498

§ 371 Date: Nov. 20, 2024

Related U.S. Application Data
(60) Provisional application No. 63/345,608, filed on May 25, 2022.

(51) Int. Cl. A61N 1/05

(52) U.S. Cl. CPC A61N 1/0573

(57) ABSTRACT

A medical device, and methods of manufacturing the same, including a fixation element having varied cross-sectional dimension. The device including a body portion and the fixation element coupled to a distal body end and extending therefrom. The fixation element is configured to affix the body portion to a wall of a heart. The fixation element defines a helical shape extending between a distal fixation end and a proximal fixation end along a direction of a helical axis. The fixation element defines a proximal fixation section proximate the proximal fixation end, a distal fixation section proximate the distal fixation end, and a middle fixation section located between the proximal and distal fixation sections. A cross-sectional dimension of the middle fixation section is smaller than a cross-sectional dimension of the proximal fixation section.

This application claims the benefit of U.S. Provisional Patent Application Ser. No. 63/345,608 filed on May 25, 2022, which is incorporated by reference herein in its entirety.

This disclosure generally relates to medical devices, such as implantable stimulation leads, including a helix fixation element and methods of the same.

Implantable medical devices (IMDs), such as implantable pacemakers, cardioverters, defibrillators, or pacemaker-cardioverter-defibrillators, provide therapeutic electrical stimulation to the heart. IMDs may provide pacing to address bradycardia, or pacing or shocks in order to terminate tachyarrhythmia, such as tachycardia or fibrillation. In some cases, the medical device may sense intrinsic depolarizations of the heart, detect arrhythmia based on the intrinsic depolarizations (or absence thereof), and control delivery of electrical stimulation to the heart if arrhythmia is detected based on the intrinsic depolarizations.

Often, the helical fixation element is formed from a segment of round diameter wire that is wound into a helical shape and assists in coupling or anchoring the medical device into cardiac tissue. However, the constant round diameter of the helical fixation element may have many limitations. Therefore, it may be beneficial for the helical fixation element to include various features to improve the coupling and/or anchoring of the medical device, as well as long-term integrity and performance.

### SUMMARY

The techniques of the present disclosure generally relate to helical fixation elements, and methods of manufacturing the same, that assist in coupling or anchoring an implantable medical device into cardiac tissue. Specifically, the helical fixation element may have a varying cross-sectional dimension along its length to optimize performance (e.g., fixation improvement, reduced trauma, longevity or fatigue performance, customizable properties, etc.) while still being formed from a single component.

For example, the helical fixation element may be stiff and sharp at the point of engagement with the cardiac tissue (e.g., at a distal end) and may be stiff for more robust attachment to a body of the implantable medical device (e.g., at a proximal end). On the other hand, a section between the ends of the helical fixation element may be more flexible for ease of maneuvering and easier removal of the delivery sheath after deployment. In other words, an intermediate section of the helical fixation element may have more bending flexibility than sections located proximate the distal and proximal ends. The variable flexibility may be achieved by adjusting a cross-sectional dimension of the helical fixation element along the length of the element. For example, the intermediate section of the helical fixation element may have a smaller cross-sectional dimension than a cross-sectional dimension proximate the distal and proximal ends. Also, the fixation element may have any number sections having a variety of different cross-sectional dimensions.

Additionally, in one or more embodiments, the helical fixation element may define a non-symmetrical cross-sectional shape by rotating the element bout the element's centroidal axis to result in a similar effect as a varying cross-section. A varying flexibility or stiffness may be provided as a function of the moment of area resulting from the rotation of the cross-sectional shape rotated about the centroidal axis.

In one or more embodiments, the helical fixation element may be cut (e.g., laser cut, water jet, plasma, etc.) from a tubing to create a varying sectional width. In other embodiments, a sheet of material may be cut (e.g., roller, laser cut, water jet, plasma, etc.) into ribbon lengths having varying sectional width and then the ribbon lengths may be formed into a helix. Further, in other embodiments, an asymmetrical cross-sectioned ribbon or element may be formed by winding into the helical form while rotating the ribbon/element about the centroidal axis.

Further, in one or more embodiments, the distal tip of the helical fixation element may include additional features to increase the tip-to-tissue anchoring, to provide auto-rotation loosening prevention, to form reservoirs for steroid elution, to increase pacing surface area, etc. In one or more embodiments, these features may be formed by processes including, e.g., grinding, electro-discharge machining, chemical etching, electro-polishing, laser ablation, forging, or other forming operations. Furthermore, in one or more embodiments, these features may also be cut during the same process in which the varying section width is cut. Also, the features may take various forms including, for example, a plurality of openings, textured surface, protrusions, ribs, scales, etc. Further yet, in one or more embodiments, the helical fixation element may include connectors or struts near the proximal end of the helical fixation element to provide more robust and redundant attachment to the body of the medical device to provide, e.g., improved fatigue performance, structural rigidity, assistance in attachment of the element to the device, etc.

One illustrative implantable medical device may include a body portion and a fixation element. The body portion may extend between a distal body end and a proximal body end. The fixation element may be coupled to the distal body end and may extend away from the body portion. The fixation element may be configured to affix the body portion to a wall of a heart. The fixation element may define a helical shape extending between a distal fixation end and a proximal fixation end along a direction of a helical axis. The proximal fixation end may be coupled to the distal body end. The fixation element may define a proximal fixation section proximate the proximal fixation end, a distal fixation section proximate the distal fixation end, and a middle fixation section located between the proximal and distal fixation sections. A cross-sectional dimension of the middle fixation section may be smaller than a cross-sectional dimension of the proximal fixation section. The medical device may also include a connector connected between adjacent portions of the fixation element spaced apart along the helical axis. The connector may include a same material as the fixation element.

One illustrative method of manufacturing a fixation element for an implantable medical device may include providing a tube defining a passage therethrough and cutting a helical shape from the tube to form a fixation element. The fixation element may extend between a distal fixation end and a proximal fixation end along a direction of a helical axis. The fixation element may define a proximal fixation section proximate the proximal fixation end, a distal fixation section proximate the distal fixation end, and a middle fixation section located between the proximal and distal fixation sections. Cutting the helical shape may include cutting a smaller cross-sectional dimension for the middle fixation section than a cross-sectional dimension of the proximal fixation section. The method may also include cutting a connector from the tube that extends between adjacent portions of the fixation element spaced apart along the helical axis.

Another illustrative implantable medical device may include a body portion extending between a distal body end and a proximal body end, and a fixation element coupled to the distal body end and extending away from the body portion. The fixation element may be configured to affix the body portion to a wall of a heart. The fixation element may define a helical shape extending between a distal fixation end and a proximal fixation end along a direction of a helical axis. The proximal fixation end may be coupled to the distal body end. The fixation element may define a proximal fixation section proximate the proximal fixation end, a distal fixation section proximate the distal fixation end, and a middle fixation section located between the proximal and distal fixation sections. A cross-sectional dimension of the middle fixation section may be smaller than a cross-sectional dimension of the proximal fixation section. The fixation element may include a plurality of features located on a surface of the fixation element proximate the distal fixation end.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram of a cardiac therapy system including an illustrative implantable medical device implanted in a patient's heart and a separate medical device positioned outside of the patient's heart.

FIG. 2 is a conceptual diagram of the illustrative implantable medical device of FIG. 1 having a helical fixation element.

FIG. 3 is a perspective view of an illustrative helical fixation element having a varying cross-section and coupled to a body of an implantable medical device according to the present disclosure.

FIG. 4 is a plan view of a helical fixation element having a varying cross-section and extending along a two-dimensional plane, and includes expanded views of different portions of the helical fixation element.

FIG. 5A is an illustrative distal tip of a helical fixation element.

FIG. 5B is another illustrative distal tip of a helical fixation element.

FIG. 5C is yet another illustrative distal tip of a helical fixation element.

FIG. 5D is yet another illustrative distal tip of a helical fixation element.

FIG. 5E is yet another illustrative distal tip of a helical fixation element.

FIG. 6A is a perspective view of an illustrative helical fixation element having a connector between adjacent portions thereof.

FIG. 6B is a perspective view of an illustrative helical fixation element having a connector, according to another embodiment, between adjacent portions thereof.

FIG. 6C is a perspective view of the helical fixation element of FIG. 6B having the connector in a more compressed configuration.

FIG. 7 is a conceptual diagram of a tube from which an illustrative helical fixation element is formed.

FIG. 8A is a conceptual diagram of a sheet from which an illustrative helical fixation element is formed along a first direction.

FIG. 8B is a conceptual diagram of a sheet from which an illustrative helical fixation element is formed along a second direction.

FIG. 9 is a flow diagram illustrating a method of manufacturing a fixation element for an implantable medical device.

FIG. 10 is conceptual diagram of a cross-section of an illustrative helical fixation element shown rotated about a centroidal axis in broken lines.

FIG. 11 is a conceptual diagram of a cross-section of an illustrative helical fixation element showing one-half of the subsequent portions of the helical fixation element at various angles.

### DETAILED DESCRIPTION

The present disclosure generally describes systems, and methods of manufacturing the same, including implantable medical devices having a helical fixation element. The helical fixation element may define a varying cross-sectional dimension along the length of the fixation element. By varying the cross-sectional dimension of the helical fixation element, the fixation element may maintain a stiff and robust distal end (e.g., to pierce into tissue) and proximal end (e.g., to attach to a body of the helical fixation element), while the length of the fixation element therebetween may remain flexible and maneuverable. For example, the middle section of the fixation element may define a smaller cross-sectional dimension than a cross-sectional dimension proximate the distal and proximal ends.

The present disclosure may also generally describe systems, and methods of manufacturing the same, including implantable medical devices having a helical fixation element defining asymmetrical cross-section dimensions along the length of the fixation element. By varying the rotation of the cross-section about the section's centroidal axis and by virtue of the second moment area of the section presented to the prevailing loads, the fixation element may maintain a stiff and robust distal end (e.g., to pierce into tissue) and proximal end (e.g., to attach to a body of the helical fixation element), while the length of the fixation element therebetween may remain flexible and maneuverable. For example, the middle section of the fixation element may define a more rotated cross-section relative to its maximum and minimum cross-sectional dimensions relative to rotation of the maximum and minimum dimension of the cross-section proximate the distal and proximal ends.

Further, the helical fixation element may include a connector between adjacent portions of the fixation element (e.g., adjacent portions that are successive turns and spaced apart). The connectors may be flexible (e.g., compressible and extendible) to help relieve some of the stress on the fixation element. Specifically, the connectors may relieve any increased stress presented from the varying cross-sectional dimension or rotated asymmetrical cross-sectional dimension of the fixation element. Further, the connectors may define at least one opening within the connector to increase the flexibility thereof. In one or more embodiments, the connector may be formed from (e.g., made of) the same material as the remainder of the fixation element. In other words, the connector may be one continuous piece with the remainder of the fixation element. In other embodiments, the connector may be formed from (e.g., made of) a second material that is different than a first material of the fixation element to, for example, confer additional utility/optimization (e.g., a second material may be radio-opaque for better fluoro-imaging).

Additionally, the helical fixation element may also include a plurality of features located proximate the distal tip of the helical fixation element. Specifically, the plurality of features may include openings, texture, barbs, etc. The features may assist in maintaining the fixation element within tissue by, e.g., restricting removal of the fixation element and/or allowing for ingrowth of tissue.

In one or more embodiments, the helical fixation element may be manufactured by cutting a tube structure into the shape of a helical structure. The connectors may also be cut from the tube structure such that the connectors are continuous with the remainder of the fixation element. In other embodiments, the fixation element may be cut from a sheet material in a two-dimensional plane. After each fixation element is cut from the sheet material, the fixation element may be formed into a helical shape.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

It will be apparent to one skilled in the art that elements or processes from one embodiment may be used in combination with elements or processes of the other embodiments, and that the possible embodiments of such devices and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

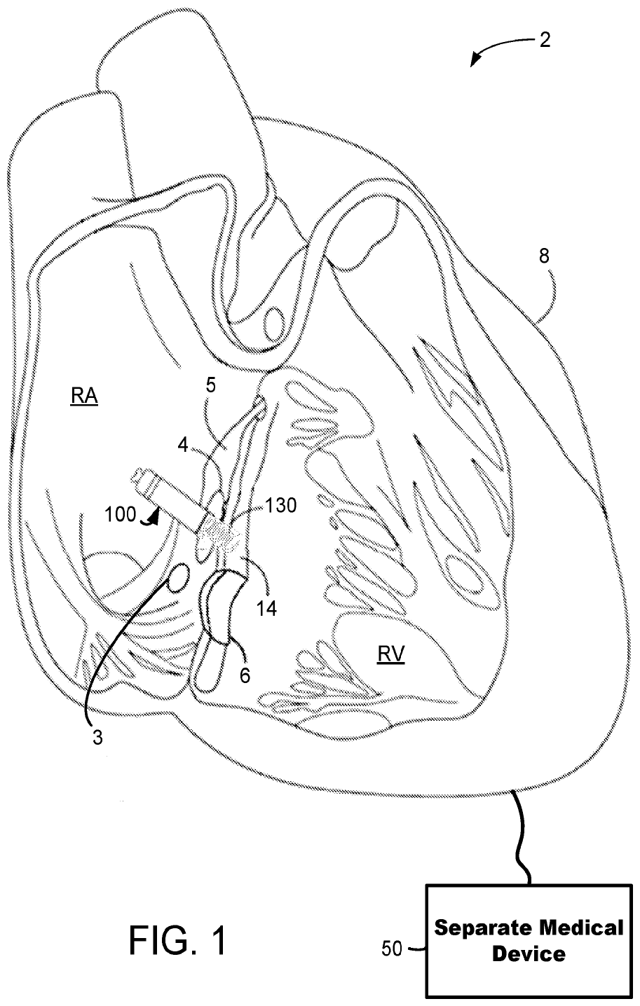

One illustrative implantable medical device 100 that may be used, at least, to treat heart conditions by delivering electrical stimulation to the AV (atrioventricular) node, nerves innervating the AV node, to other portion(s) of the right and/or left atrium, and/or to the right and/or left ventricle (or to multiple chambers of the heart) is illustrated in FIG. 1. Although it is to be understood that the present disclosure may utilize one or both of leadless and leaded implantable medical devices, the illustrative cardiac therapy system of FIG. 1 includes a leadless intracardiac medical device 100 implanted in a patient's heart 8. Furthermore, although the device 100 is configured to deliver electrical stimulation to the AV node or nerves innervating the AV node as described herein, in some embodiments, the device 100 may be configured for single chamber pacing and may, for example, switch between single chamber and multiple chamber pacing (e.g., dual or triple chamber pacing). As used herein, “intracardiac” refers to a device configured to be implanted entirely within a patient's heart, for example, to provide cardiac therapy. Further, it is contemplated herein that the device could include epicardial positioning or ventricle from atrium (VfA) positioning. Further yet, in other embodiments, it is contemplated that the device may include neuro stimulation devices, drug delivery devices, etc.

The device 100 is shown implanted in the right atrium (RA) of the patient's heart 8 in a target implant region 4. The device 100 may include one or more fixation elements 130 that anchor a distal end of the device 100 against the atrial endocardium in a target implant region 4 (e.g., within the triangle of Koch region). In other words, the fixation element 130 may be securely anchored into the tissue for stabilizing the implant position of the device 100. In one or more embodiments, the target implant region 4 may lie between the Bundle of His 5 and the coronary sinus 3 and may be adjacent, or next to, the tricuspid valve 6. As such, the device 100 may be described as a right atrial-implanted device as it is disposed in the right atrium. While a specific target implant region 4 is shown in FIG. 1, other implant locations and configurations are contemplated herein, for example, including within either the right or left atrium, or within either the right or left ventricle.

In one or more embodiments, the device 100 may be configured to sense electrical activity of the heart, including electrical activity originating in or conducted via the cardiac conduction system, and/or nerve activity (e.g., parasympathetic nerve activity) of one or both of the AV node or nerves innervating the AV node (e.g., including different bundles of the AV node) using one or more electrodes located, for example, proximate endocardial tissue of the right atrium within the triangle of Koch, or alternatively within any of the other chambers of the heart or in other locations within the right atrium. The electrode(s), as described further herein, may be positioned adjacent the endocardial tissue of the right atrium within the triangle of Koch, or in any other location described herein, utilizing the fixation element(s) 130. In at least one embodiment, the electrode(s) are positioned adjacent the AV nodal fatty pad in the right atrium. In at least one embodiment, the fixation element(s) (or portion(s) thereof) may serve as electrode(s) in addition to performing the fixation function.

The device 100 may be described as a leadless implantable medical device. As used herein, “leadless” refers to a device being free of a lead extending out of the patient's heart 8. Further, although a leadless device may have a lead, the lead would not extend from outside of the patient's heart to inside of the patient's heart or would not extend from inside of the patient's heart to outside of the patient's heart. Some leadless devices may be introduced through a vein, but once implanted, the device is free of, or may not include, any transvenous lead and may be configured to provide cardiac therapy without using any transvenous lead. Further, a leadless device, in particular, does not use a lead to operably connect to one or more electrodes when a housing of the device is positioned in the atrium. Additionally, a leadless electrode may be coupled to the housing of the medical device without using a lead between the electrode and the housing.

The device 100 may be configured to monitor one or more physiological parameters of a patient (e.g., electrical activity of a patient's heart including electrical activity originating in or conducted via the cardiac conduction system, chemical activity of a patient's heart, hemodynamic activity of a patient's heart, motion of a patient's heart including contraction of one or more chambers thereof, electrical activity of the AV node and/or nerves innervating the AV node, pressure, oxygen level, temperature, etc.). The monitored physiological parameters, in turn, may be used by the IMD to detect various cardiac conditions including, e.g., ventricular tachycardia (VT), ventricular fibrillation (VF), supraventricular ventricular tachycardia (SVT), atrial fibrillation (AF), atrial tachycardia (AT), myocardial ischemia/infarction, etc., and to treat such cardiac conditions with therapy. Such therapy may include delivering electrical stimulation to the cardiac conduction system and/or to the myocardium in one or more heart chambers, to the AV node or nerves (e.g., nerve tissue) innervating the AV node within the triangle of Koch region of the right atrium, electrical stimulation for pacing the patient's heart (e.g., bradycardia pacing, cardiac resynchronization therapy, anti-tachycardia pacing (ATP), and/or other pacing therapies), etc. Further, in at least one embodiment, the device 100 may be capable of delivering high-energy shock pulses for cardioversion/defibrillation therapy delivered in response to, e.g., tachycardia detections.

The device 100 may include a plurality of electrodes. One or more of the electrodes may be configured to deliver stimulation (e.g., such as AV nodal stimulation) to cause contraction of one or more heart chambers, and/or sense cardiac electrical activity. The electrodes may be able to sense electrical activity of the patient's heart including conduction system activity as well as depolarizations of the heart tissue, to deliver pacing therapy to cardiac tissue to induce depolarization of cardiac tissue, and/or to deliver cardioversion shocks to cardiac tissue.

In one or more embodiments, the cardiac therapy system 2 may also include a separate medical device 50 (depicted diagrammatically in FIG. 1), which may be positioned outside the patient's heart 8 (e.g., subcutaneously) and may be operably coupled to the patient's heart 8 to deliver cardiac therapy thereto. In one example, separate medical device 50 may be an extravascular ICD. In some embodiments, an extravascular ICD may include a defibrillation lead including, or carrying, a defibrillation electrode. A therapy vector may exist between the defibrillation electrode on the defibrillation lead and a housing electrode of the ICD. Further, one or more electrodes of the ICD may also be used for sensing electrical signals related to the patient's heart 8. The ICD may be configured to deliver shock therapy including one or more defibrillation or cardioversion shocks. For example, if an arrhythmia is sensed, the ICD may send a pulse via the electrical lead wires to shock the heart and restore its normal rhythm. In some examples, the ICD may deliver shock therapy without placing electrical lead wires within the heart or attaching electrical wires directly to the heart (subcutaneous ICDs). Examples of extravascular, subcutaneous ICDs that may be used with the system 2 described herein may be described in U.S. Pat. No. 9,278,229 (Reinke et al.), issued 8 Mar. 2016, which is incorporated herein by reference in its entirety.

In the case of shock therapy (e.g., defibrillation shocks provided by the defibrillation electrode of the defibrillation lead), the separate medical device 50 (e.g., extravascular ICD) may include a control circuit that uses a therapy delivery circuit to generate defibrillation shocks having any of a number of waveform properties, including leading-edge voltage, tilt, delivered energy, pulse phases, and the like. The therapy delivery circuit may, for instance, generate monophasic, biphasic, or multiphasic waveforms. Additionally, the therapy delivery circuit may generate defibrillation waveforms having different amounts of energy. For example, the therapy delivery circuit may generate defibrillation waveforms that deliver a total of between approximately 60-80 Joules (J) of energy for subcutaneous defibrillation.

The separate medical device 50 may further include a sensing circuit. The sensing circuit may be configured to obtain electrical signals sensed via one or more combinations of electrodes and to process the obtained signals. The components of the sensing circuit may include analog components, digital components, or a combination thereof. The sensing circuit may, for example, include one or more sense amplifiers, filters, rectifiers, threshold detectors, analog-to-digital converters (ADCs), or the like. The sensing circuit may convert the sensed signals to digital form and provide the digital signals to the control circuit for processing and/or analysis. For example, the sensing circuit may amplify signals from sensing electrodes and convert the amplified signals to multi-bit digital signals by an ADC, and then provide the digital signals to the control circuit. In one or more embodiments, the sensing circuit may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to the control circuit.

The device 100 and the separate medical device 50 may cooperate to provide cardiac therapy to the patient's heart 8. For example, the device 10 and the separate medical device 50 may be used to detect tachycardia, monitor tachycardia, and/or provide tachycardia-related therapy. For example, the device 100 may communicate with the separate medical device 50 wirelessly to trigger shock therapy using the separate medical device 50. As used herein, “wirelessly” refers to an operative coupling or connection without using a metal conductor between the device 100 and the separate medical device 50. In one example, wireless communication may use a distinctive, signaling, or triggering electrical pulse provided by the device 100 that conducts through the patient's tissue and is detectable by the separate medical device 50. In another example, wireless communication may use a communication interface (e.g., an antenna) of the device 100 to provide electromagnetic radiation that propagates through patient's tissue and is detectable, for example, using a communication interface (e.g., an antenna) of the separate medical device 50.

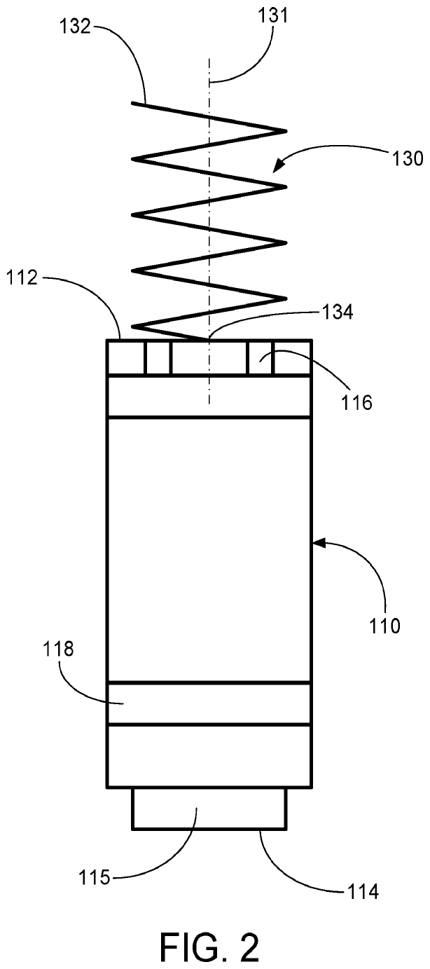

FIG. 2 is an enlarged conceptual diagram of the implantable medical device 100 of FIG. 1. In particular, the device 100 is configured for treating heart conditions through sensing cardiac signals and/or delivering pacing therapy (e.g., for single or multiple chamber cardiac therapy). The medical device 100 may include a housing or body portion 110. The body portion 110 may define a hermetically-sealed internal cavity in which internal components of the device 100 reside, such as a sensing circuit, therapy delivery circuit, control circuit, memory, telemetry circuit, other optional sensors, and a power source. The body portion 110 may include (e.g., be formed of or from) an electrically conductive material such as, e.g., titanium or titanium alloy, stainless steel, MP35N (a non-magnetic nickel-cobalt-chromium-molybdenum alloy), platinum alloy, or other bio-compatible metal or metal alloy. In other examples, the body portion 110 may include (e.g., be formed of or from) a non-conductive material including ceramic, glass, sapphire, silicone, polyurethane, epoxy, acetyl co-polymer plastics, polyether ether ketone (PEEK), a liquid crystal polymer, or other biocompatible polymer.

In at least one embodiment, the body portion 110 of the implantable medical device 100 may be described as extending between a distal body end 112 and a proximal body end 114. Further, the body portion 110 may define a generally-cylindrical shape, e.g., to facilitate catheter delivery. In other embodiments, the body portion 110 may be prismatic or any other shape to perform the necessary functionality and utility. The body portion 110 may include a delivery tool interface member 115, e.g., defined, or positioned, at the proximal body end 114, for engaging with a delivery tool during implantation of the device 100.

All or a portion of the body portion 110 may function as a sensing and/or pacing electrode during cardiac therapy. For example, in one or more embodiments, the body portion 110 may include a proximal housing-based electrode 118 that circumscribes a proximal portion (e.g., closer to the proximal body end 114 than the distal body end 112) of the body portion 110. In other examples, however, the proximal housing-based electrode 118 may be located at other positions along the body portion 110, e.g., more distal relative to the position shown. In support of pacing and/or sensing functions, the proximal housing-based electrode 118 may serve as a return electrode or return anode, and other electrode(s), some or all of which may contact tissue, may serve as cathodes to deliver pacing pulses to the tissue and/or sense electrical activity.

When the body portion 110 is (e.g., defines, formed from, etc.) an electrically-conductive material, such as a titanium alloy or other examples listed above, portions of the body portion 110 may be electrically insulated by a non-conductive material, such as a coating of parylene, polyurethane, silicone, epoxy, or other biocompatible polymer, leaving one or more discrete areas of conductive material exposed to form, or define, the proximal housing-based electrode 118. When the body portion 110 is (e.g., defines, formed from, etc.) a non-conductive material, such as a ceramic, glass or polymer material, an electrically-conductive coating or layer, such as a titanium, platinum, stainless steel, or alloys thereof, may be applied to one or more discrete areas of the body portion 110 to form, or define, the proximal housing-based electrode 118. In other examples, the proximal housing-based electrode 118 may be a component, such as a ring electrode, that is mounted or assembled onto the body portion 110. The proximal housing-based electrode 118 may be electrically coupled to internal circuitry of the device 100, e.g., via the electrically-conductive body portion 110 or an electrical conductor when the body portion 110 is a non-conductive material.

Furthermore, the implantable medical device 100 may include a fixation element 130 coupled to the distal body end 112 of the body portion 110 and extending away from the body portion 110. The fixation element 130 may act as an anchor and may be configured to affix or attach the body portion 110 to a wall of the heart (e.g., to cardiac tissue). Also, in one or more embodiments, all or a portion of the fixation element 130 may also act as a tissue piercing electrode (e.g., piercing through one or more tissue layers). The tissue piercing electrode (e.g., for delivering of pacing energy to and/or sensing signals from the tissue) may be located at or near the distal end of the fixation element 130 (e.g., a tip electrode).

The fixation element 130 may define a helical shape extending between a distal fixation end 132 (e.g., a distal tip) and a proximal fixation end 134 along a direction of a helical axis 131. In other words, the fixation element 130 may include a plurality of turns defining a radius and pitch to form a helical shape. The proximal fixation end 134 of the fixation element 130 may be coupled to the distal body end 112 of the body portion 110. Further, the fixation element 130 may be coaxial with the body portion 110. The fixation element 130 may include (e.g., be formed of) any suitable material. For example, the fixation element 130 may include noble metal alloys, platinum-iridium, platinum, stainless steel, tantalum alloys and niobium alloys, elgiloy, cobalt-chromium alloy, MP35N alloy, etc. Specifically, the fixation element 130 may include nickel and cobalt-free austenitic stainless steel (e.g., nitrogen stabilized). Furthermore, in one or more embodiments, the fixation element 130 may include alloy compositions specifically selected to confer properties of springiness and resilience to avoid plastic damage during clinical implant and subsequent in-service loads. In one or more embodiments, the fixation element 130 may be covered with an electrically insulating material, coating or surface treatment except where an electrode is present (e.g., at the tip of the fixation element 130 and/or at any other location of the fixation element 130 that serves as an electrode).

In one or more embodiments, one or multiple non-tissue piercing electrodes 116 (e.g., housing-based electrodes) may be provided at (e.g., along a periphery of) the distal body end 112 of the body portion 110. The non-tissue piercing electrodes 116 may operate to sense electrical activity at and/or deliver electrical stimulation to any suitable location in the heart, including within the right atrium, such as one or both of the AV node or nerves innervating the AV node. The non-tissue piercing electrode(s) 116 may be formed of an electrically conductive material, such as copper, platinum, iridium, or alloys thereof. Specifically, the non-tissue piercing electrodes 116 may be spaced apart radially at equal distances along the outer periphery of the distal body end 112 and may include any suitable number of non-tissue piercing electrode(s) 116. When the fixation element 130 is advanced into the cardiac tissue, at least one non-tissue piercing electrode 116 may be positioned against, in intimate contact with, or in operative proximity to, a cardiac tissue surface for delivering AV nodal stimulation and/or sensing nerve activity from one or both of the AV node or nerves innervating the AV node. For example, non-tissue piercing electrode(s) 116 may be positioned in contact with right atrial endocardial tissue for stimulation (such as AV nodal stimulation) and electrical activity sensing. The non-tissue piercing electrode(s) 116 may be fixed in position with respect to the body portion 110 or, alternatively, elastically biased in a direction (e.g., longitudinally) away from the body portion so as to maintain firm contact with adjacent cardiac tissue as the heart and/or device 100 move. Such elastically biased electrode(s) may have a “ramp” configuration as shown in U.S. Patent Application No. 2020/0398045 A1 (e.g., the second electrode 28 described therein and shown in FIGS. 2-6D), which is incorporated herein by reference in its entirety.

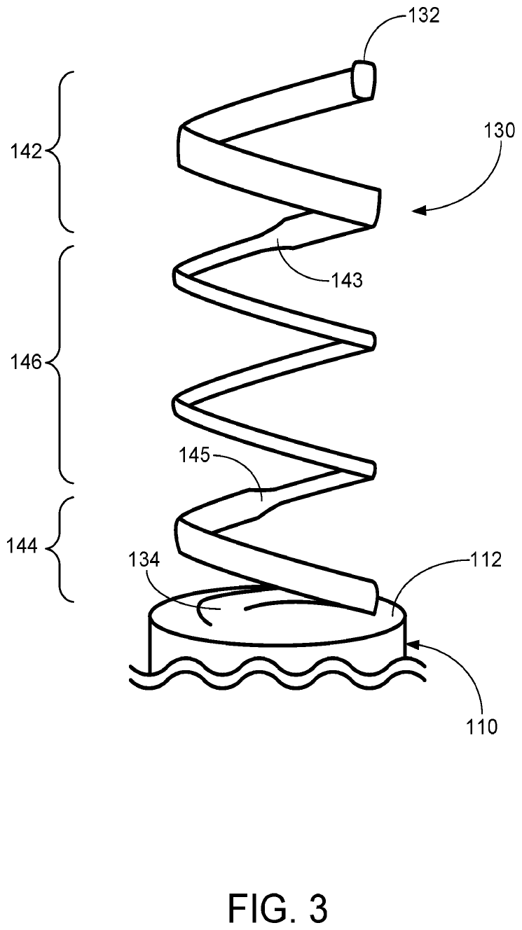

FIG. 3 illustrates an expanded view of the fixation element 130 coupled to the body portion 110 showing a varying dimension along the length of the fixation element 130. By varying the cross-sectional dimension along the length of the fixation element 130, the fixation element 130 may have different characteristics or properties at those sections. For example, a fixation element 130 having a wider cross-sectional dimension proximate the distal fixation end 132 that narrows towards the proximal fixation end 134 may help to minimize tissue damage. In other words, while the distal fixation end 132 of the fixation element 130 may define a sharp distal tip for piercing tissue, the portion thereafter (e.g., near the distal fixation end 132) may define a relatively wider cross-sectional dimension (e.g., compared to portions more proximal) to maintain stiffness and rigidity. Further, the cross-sectional dimension of the fixation element 130 may narrow towards the proximal direction to reduce potential damage to the tissue (e.g., by minimizing the footprint of the fixation element 130).

Also, for example, a fixation element 130 having a wider cross-sectional dimension proximate the proximal fixation end 134 that narrows towards the distal fixation end 132 may help provide strain relief at the base. In other words, providing a wider base of the fixation element 130 at the connection point with the body portion 110 (e.g., compared to the remainder of the fixation element 130) may maintain stiffness and more robust attachment.

It is noted that a varying cross-sectional dimension of the fixation element 130 may be defined by any corresponding dimension along the fixation element 130. For example, if the fixation element 130 defines a circular cross-section, the varying cross-sectional dimension may be a diameter of the circular shape. Also, for example, if the fixation element 130 defines a rectangular cross-section, the varying cross-sectional dimension may be the same dimension at various points along the fixation element 130. Specifically, the varying cross-sectional dimension may relate to a width of the fixation element 130 even if a thickness of the fixation element 130 remains constant. In some embodiments, multiple dimensions of the cross-sectional shape of the fixation element 130 may vary.

The fixation element 130 may be divided into sections to define the varying cross-sectional dimensions. For example, as shown in FIG. 3, the fixation element 130 may define a proximal fixation section 144 proximate the proximal fixation end 134, a distal fixation section 142 proximate the distal fixation end 132, and a middle fixation section 146 located between the proximal and distal fixation sections 144, 142. As described herein, in one or more embodiments, a cross-sectional dimension of the middle fixation section 146 of the fixation element 130 may be smaller than a cross-sectional dimension of the proximal fixation section 144. Also, in one or more embodiments, the cross-sectional dimension of the middle fixation section 146 may be smaller than a cross-sectional dimension of the distal fixation section 142. In other embodiments, the cross-sectional dimension of the middle fixation section 146 may be larger than a cross-sectional dimension of one or both of the distal and proximal fixation sections 142, 144. In yet other embodiments, two of the sections 142, 144, 146 may define a similar cross-sectional dimension that may be different than the third section (e.g., larger or smaller).

As shown in FIG. 3, the fixation element 130 may combine the concepts of a larger cross-sectional dimension within the distal fixation section 142 and the proximal fixation section 144, and a smaller cross-sectional dimension within the middle fixation section 146 (e.g., relative to one another). By combining these cross-sectional dimensions (e.g., narrower in the middle of the fixation element 130 as compared to the distal and proximal ends), the fixation element 130 may be stiffer and more robust at the point in which the fixation element 130 pierces tissue (e.g., the distal fixation end 132) and connects to the body portion 110 (e.g., the proximal fixation end 134), but also may be more flexible and maneuverable due to the narrower middle fixation section 146. Specifically, the cross-sectional dimension of the middle fixation section 146 may define a width of about 0.3 millimeters (e.g., 0.012 inches), the cross-sectional dimension of the proximal fixation section 144 may define a width of about 0.5 millimeters (e.g., 0.02 inches), and the cross-sectional dimension of the distal fixation section 142 may define a width of about 0.5 millimeters (e.g., 0.02 inches). More specifically, any of the sections 142, 144, 146 may define cross-sectional dimension or width of about 0.1 millimeter (e.g., 0.004 inches) to about 0.7 millimeters (e.g., 0.028 inches).

Further, the fixation element 130 may include various combinations of cross-sectional dimensions depending on the specific application of the fixation element 130. For example, in one or more embodiments, the cross-sectional dimension of the distal fixation section 142 may be smaller than the cross-sectional dimension of the proximal fixation section 144 (and/or middle fixation section 146). As such, the middle and/or proximal section 146, 144 may be more stiff while the distal section 142 may be more flexible (e.g., if the heart tissue is more fragile). Further yet, the fixation element 130 may include any number of sections having different cross-sectional dimensions. For example, the fixation element 130 may include two sections having different cross-sectional dimensions. In other embodiments, the fixation element 130 may include four, five, six, etc. sections having different cross-sectional dimensions (e.g., between adjacent sections). For example, in one or more embodiments, the fixation element 130 may define two different cross-sectional dimensions that alternate between sections of the fixation element 130.

The sections 142, 144, 146 of the fixation element 130 may extend for any suitable length along the fixation element 130. For example, the lengths of the sections 142, 144, 146 of the fixation element 130 may be customizable depending on the specific application to, e.g., amplify or reduce the feature of that particular section. In one or more embodiments, the distal fixation section 142, the proximal fixation section 144, and the middle fixation section 146 may define a same length measured along the fixation element 130 (e.g., each section 142, 144, 146 may be one-third of the length of the fixation element 130). In one or more embodiments, the fixation element 130 may include a longer section at the base for attachment to the device 100 such that the proximal fixation section 144 may extend for one-half of the fixation element 130, and the distal fixation section 142 and the middle fixation section 146 may extend for one-quarter of the fixation element 130 each. In other embodiments, the fixation element 130 may include a longer section at the tip for more securement to the tissue such that the distal fixation section 142 may extend for one-half of the fixation element 130, and the proximal fixation section 144 and the middle fixation section 146 may extend for one-quarter of the fixation element 130 each.

Furthermore, the fixation element 130 may taper between the varying cross-sectional dimensions. For example, the fixation element 130 may define a taper 143 between the middle fixation section 146 and the distal fixation section 142. Also, for example, the fixation element 130 may define a taper 145 between the middle fixation section 146 and the proximal fixation section 144. Each of these tapers 143, 145 may provide a gradual progression of the varying cross-sectional dimension of the fixation element 130. Specifically, the tapers 143, 145 of the fixation element 130 may help to limit the stress concentrations to any one point along the fixation element 130.

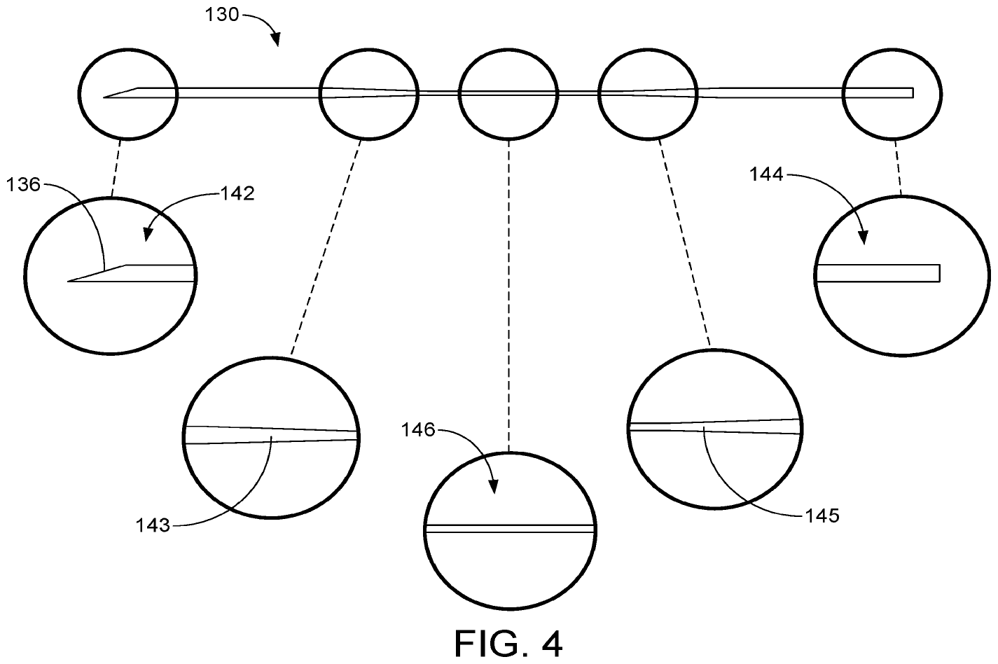

The varying cross-sectional dimension of the fixation element 130 is also shown in FIG. 4. For example, FIG. 4 illustrates a fixation element 130 extending along a plane (e.g., after being cut from a sheet material as described herein in connection with FIGS. 8A and 8B). As such, the fixation element 130 may be shaped into a plurality of pitched turns to form a helical shape as shown in FIG. 3.

As shown in FIG. 4, the distal fixation section 142 of the fixation element 130 includes a distal tip 136 that forms a point for insertion into tissue. Also, the proximal fixation section 144 and the distal fixation section 142 of the fixation element 130 define a larger cross-sectional dimension (e.g., a width) than the middle fixation section 146 of the fixation element 130. Further, as noted herein, the fixation element 130 defines a taper 143 between the middle fixation section 146 and the distal fixation section 142, and a taper 145 between the middle fixation section 146 and the proximal fixation section 144.

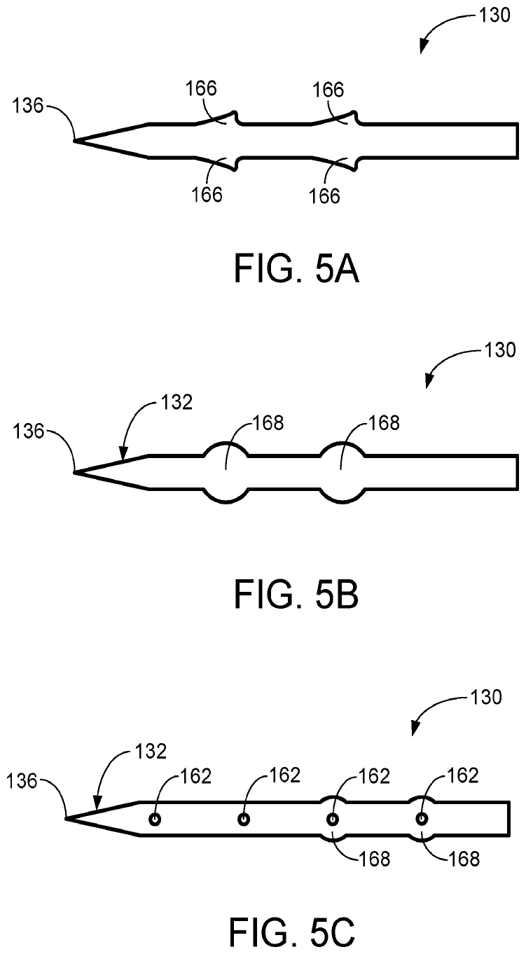

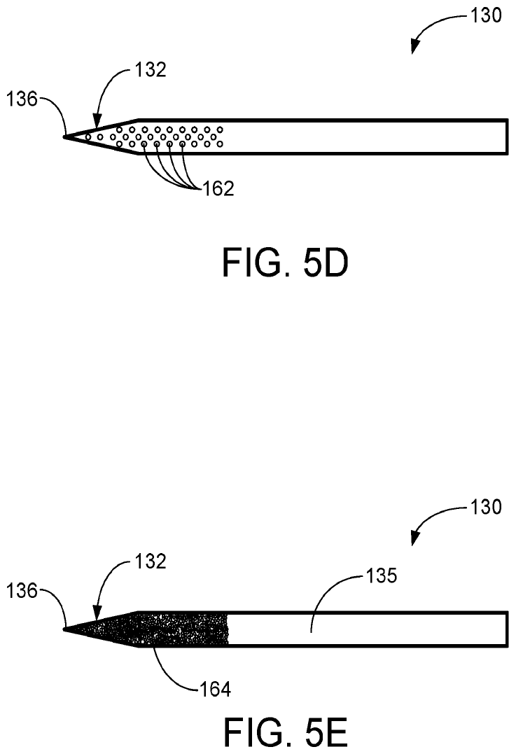

The fixation element 130 may define a variety of different types of features located on a surface of the fixation element 130 proximate the distal fixation end 132 (e.g., near the distal tip 136), e.g., as shown in FIGS. 5A-5E. When the fixation element 130 is inserted into the cardiac tissue, the features may interact with the tissue in various different ways. For example, the features of the fixation element 130 may provide additional anchoring, prevent auto-rotation, provide steroid delivery, increase pacing tip surface area, etc.

As shown in FIG. 5A, the fixation element 130 may include angled barbs 166 that assist with anchoring the fixation element 130 into the tissue. For example, the angled barbs 166 may be shaped such that the angled barbs 166 may easily enter the tissue but may provide resistance from being removed from the tissue (e.g., inadvertent device dislodgment). The fixation element 130 may include any number of angled barbs 166. As shown in FIG. 5A, the fixation element 130 includes two pairs of angled barbs 166 (e.g., on either side of the fixation element 130) at two different positions along the length of the fixation element 130.

As shown in FIG. 5B, the fixation element 130 may include round barbs 168 that assist with anchoring the fixation element 130 into the tissue. For example, the round barbs 168 may be shaped such that the round barbs 168 help provide resistance from being removed from the tissue. The fixation element 130 may include any number of round barbs 168. As shown in FIG. 5B, the fixation element 130 includes two pairs of round barbs 168 (e.g., on either side of the fixation element 130) at two different positions along the length of the fixation element 130.

As shown in FIG. 5C, the fixation element 130 may include a plurality of openings 162 extending through the fixation element 130 and round barbs 168. The round barbs 168 may be shaped to assist with anchoring the fixation element 130 into the tissue by providing resistance from being removed from the tissue. The plurality of openings 162 may also assist with anchoring the fixation element 130 into the tissue by allowing ingrowth of tissue through the openings 162 to maintain the fixation element 130 in place. Additionally, in one or more embodiments, the plurality of openings 162 may contain or capture steroids (e.g., by loading the tip area with more steroid) to be delivered into the tissue. The steroid may be applied to the fixation element 130 and within the plurality of openings 162 in a variety of different ways including, for example, being dip coated with steroid solutions, sprayed with a fine coating of steroid (e.g., mixed with a polymer), etc. There may be any number of openings 162 and round barbs 168. For example, as shown in FIG. 5C, the fixation element 130 includes four openings 162 and two round barbs 168. Also, in some embodiments (e.g., as shown in FIG. 5C), at least a portion of the openings 162 may be aligned with at least a portion of the round barbs 168. Furthermore, the openings 162 may define any suitable diameter such as, for example, about 0.12 millimeters to 0.25 millimeters (e.g., 0.005 to 0.1 inches).

As shown in FIG. 5D, the fixation element 130 may include a plurality of openings 162 extending through the fixation element 130. The plurality of openings 162 may assist with anchoring the fixation element 130 into the tissue by allowing ingrowth of tissue through the openings 162 to maintain the fixation element 130 in place. Also, in one or more embodiments, the plurality of openings 162 may contain or capture steroids (e.g., by loading the tip area with more steroid) to be delivered into the tissue. The steroid may be applied to the fixation element 130 and within the plurality of openings 162 in a variety of different ways including, for example, being dip coated with steroid solutions, sprayed with a fine coating of steroid (e.g., mixed with a polymer), etc. Further, the openings 162 may assist with increasing the surface area of the fixation element 130 within the tissue (e.g., corresponding with the pacing tip). There may be any suitable number of openings 162 and the openings 162 may be arranged in any suitable way. For example, as shown in FIG. 5D, the fixation element 130 includes three rows of offset openings 162 arranged relative to the distal tip 136. Further, the openings may define any suitable diameter such as, for example, about 0.12 millimeters to 0.25 millimeters (e.g., 0.005 to 0.1 inches).

As shown in FIG. 5E, the fixation element 130 may include a textured surface 164 proximate the distal fixation end 132 (e.g., at and/or near the distal tip 136). The textured surface 164 may assist with increasing the surface area of the fixation element 130 within the tissue (e.g., corresponding to the pacing tip). Further, the textured surface 164 may help with maintaining the position of the fixation element 130 within the tissue (e.g., acting as a plurality of smaller barbs). Further yet, the textured surface 164 may assist with minimizing auto rotation of the fixation element 130 or may assist with steroid retention if applied in this area. The textured surface 164 may define any suitable shape and size. For example, the textured surface 164 may define a microstructure on the surface 135 of the fixture element 130 that are shaped similar to fish scales, grooves, peaks, undulations, tines, waves, etc. Also, the textured surface 164 may extend for any suitable length along the fixation element 130 proximate the distal fixation end 132 and may be located on one or both sides of the fixation element 130.

Furthermore, in one or more embodiments, the fixation element 130 may include one or more flexible barbs located within the helical section. For example, one or more flexible barbs may be cut into the fixation element (e.g., at the same time the fixation element is formed). The one or more flexible barbs may be manipulated to create a bias to point the one or more flexible barbs in a slightly outward direction from the helical path. The bias of the one or more flexible barbs may be configured such that upon turning the helical section in the direction to engage the fixation element 130 with the tissue, the one more flexible barbs may flex inwards to become coincident with the fixation element (e.g., to maintain smooth progression of fixation and not impede). Upon the helical section of the fixation element encountering torques in the opposite direction (e.g., to remove the fixation element), the one or more flexible barbs may flex outward and resist further turning by engaging with the tissue (e.g., to oppose the torque applied). For example, an end (e.g., a distal end) of the one or more flexible barbs may be connected to the remainder of the fixation element (e.g., to be inserted into tissue first) and the other end (e.g., a proximal end) of the one or more flexible barbs may be free or separated from the remainder of the fixation element (e.g., so that the trailing end may flex or bias relative to the fixation element). In one or more embodiments, the fixation element 130 may define a wider section at the portion in which the one or more flexible barbs is located.

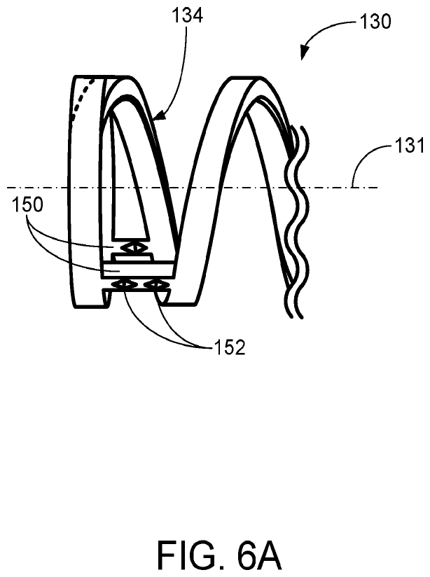

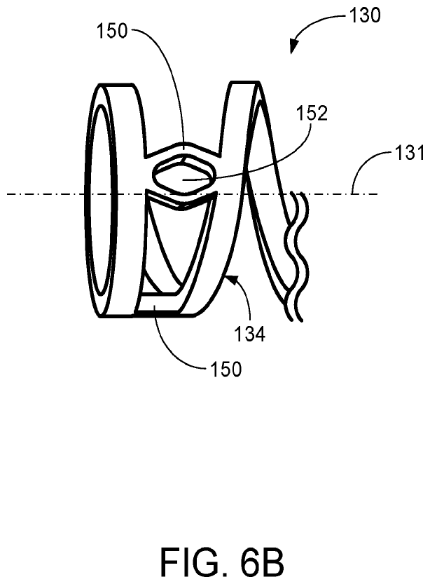

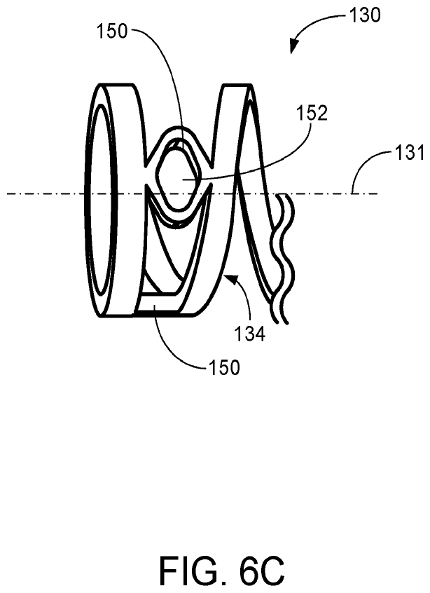

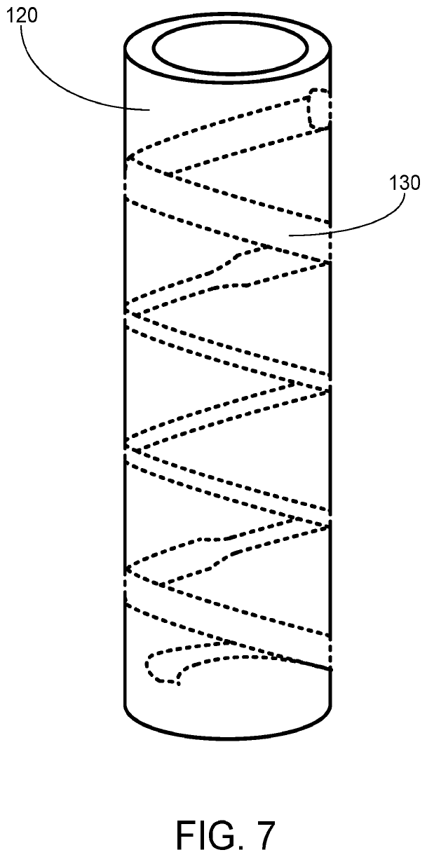

In one or more embodiments, the fixation element 130 may also include a connector 150 or strut connected between adjacent portions of the fixation element 130 spaced apart along the helical axis 131, for example, as shown in FIGS. 6A, 6B, and 6C. In combination with the varying cross-sectional dimensions of the fixation element 130 as described herein, the connector 150 may provide additional stiffness to balance out the flexibility provided by the varying cross-sectional dimensions. In other words, the connector 150 may balance stiffness and flexibility by reducing the amount of stress applied to the fixation element 130 while still allowing for some movement allowed by the varying cross-sectional dimensions of the fixation element 130. Further, the connector 150 may also provide some flexibility to the fixation element 130 by being extendible and compressible (e.g., bending along the helical axis 131) such that the connector 150 may relieve some of the stress on the helical structure. Additionally, in one or more embodiments, the connector 150 may provide a redundant and resilient connection for the fixation element 130 (e.g., redundant electrical connection points, redundant structural connection points, etc.).

The connector 150 may be connected between successive turns of the fixation element 130 along the helical axis 131. For example, the fixation element 130 may define a space or gap between successive turns and the connector 150 may span that space or gap to connect those portions of the fixation element 130. Further, the connector 150 may be positioned at any suitable location along the length of the fixation element 130. Specifically, the connector 150 may be located proximate the proximal fixation end 134 of the fixation element 130. More specifically, the connector 150 may be positioned between a base of the fixation element 130 and an adjacent portion or turn of the fixation element 130 (e.g., to locate the connector at a proximal position along the fixation element 130). Furthermore, the connector 150 may be positioned such that the fixation element 130 extends in both directions from an end of the connector 150 (e.g., from both directions at the connection point with the connector 150). In other words, the connector may not be positioned at an end or terminal point of the fixation element 130.

The connector 150 may include (e.g., be formed of) any suitable material. For example, in one or more embodiments, the connector 150 may include the same material as the remainder of the fixation element 130. Specifically, in one or more embodiments, the connector 150 may be formed or cut from the same material (and at the same time) as the fixation element 130 (e.g., as described herein). In other embodiments, the connector 150 may include a different material from the remainder of the fixation element 130. For example, the connector may include noble metal alloys, platinum-iridium, platinum, stainless steel, tantalum alloys and niobium alloys, elgiloy, cobalt-chromium alloy, MP35N alloy, nickel and cobalt-free austenitic stainless steel (e.g., nitrogen stabilized), alloy compositions specifically selected to confer properties of springiness and resilience (e.g., to avoid plastic damage during clinical implant and subsequent in-service loads), etc.

The connector 150 may provide a flexibility to the fixation element 130 in a variety of different ways. For example, in one or more embodiments, the material of the connector 150 may provide an increased flexibility. Also, in one or more embodiments, the connector 150 may define at least one opening 152 (e.g., as shown in FIGS. 6A-6C). The at least one opening 152 defined within the connector 150 may form a spring-like effect that may allow for some compressibility and flexibility of the fixation element 130. The at least one opening 152 may define any suitable shape and size. As shown in FIGS. 6A-6C, the at least one opening 152 may define a diamond shape. Further, the connector 150 may include any suitable number of openings 152. As shown in FIG. 6A, the connector 150 defines two openings 152 arranged along the helical axis 131. As shown in FIGS. 6B and 6C, the connector 150 defines one opening 152 that is larger than the openings of FIG. 6A.

Also, as shown in FIGS. 6A-6C, the connector 150 may define a variety of different suitable shapes. For example, as shown in FIG. 6A, the connector 150 defines a generally rectangular shape and may define a width of about 0.3 millimeters (e.g., 0.012 inches) to 0.5 millimeters (e.g., 0.02 inches). As shown in FIGS. 6B and 6C, the connector 150 defines a shape that follows the shape of the opening 152 to form a compressible structure (e.g., along the helical axis 131). Specifically, FIG. 6B illustrates the connector 150 in a relaxed (or uncompressed) state and FIG. 6C illustrates the connector 150 in a flexed (or at least partially compressed) state.

Additionally, the fixation element 130 may include any suitable number of connectors 150. For example, FIGS. 6A-6C illustrate two connectors 150 spaced apart from one another and connected to different portions of the fixation element. In other words, the fixation element 130 may include an additional connector connected between adjacent portions of the fixation element 130 along the helical axis 131 and spaced apart from the other connector.

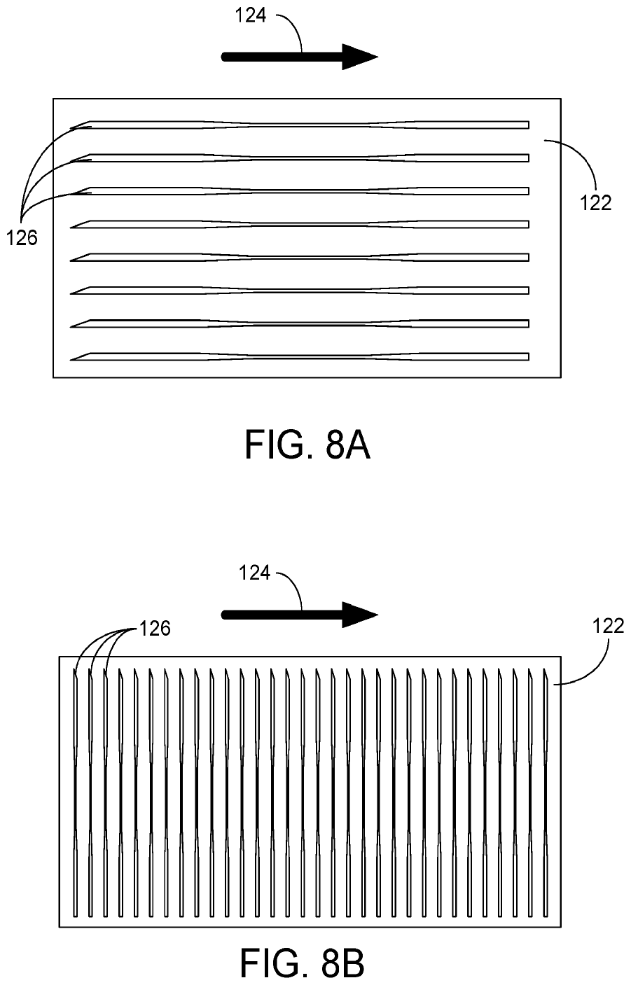

The fixation elements 130 may be manufactured in a variety of different ways. For example, the fixation elements 130 may be created through a process of cutting (e.g., laser cutting) the shape of the fixation element 130 from a tube structure or cutting (e.g., laser cutting) the shape of the fixation element 130 from a sheet material and formed into a helical shape thereafter. For example, FIG. 7 illustrates a tube 120 from which the fixation element 130 (illustrated in broken lines) may be cut. The tube structure 120 may define a thickness that defines the thickness of the fixation element 130. However, the tube structure 120 may be cut in such a way to vary the width of the resulting fixation element 130 (e.g., while the fixation element 130 maintains the thickness defined by the tube 120).

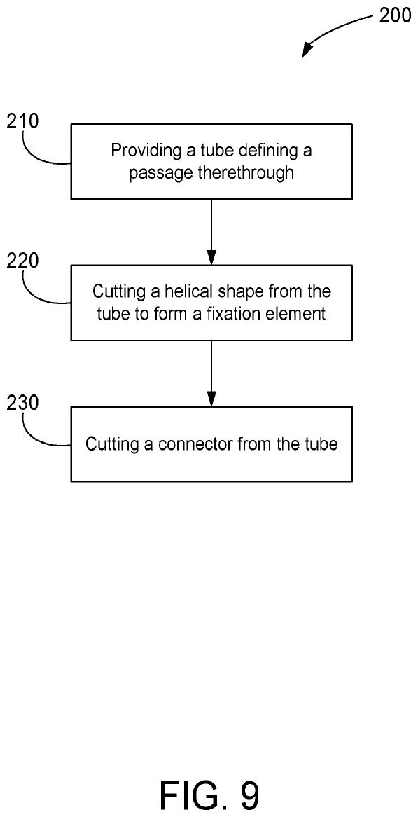

The method of manufacturing a fixation element 130 for an implantable medical device is illustrated in the flow diagram of FIG. 9. For example, the method 200 may include providing 210 a tube (e.g., as shown in FIG. 7) defining a passage therethrough. The method 200 may also include cutting 220 a helical shape from the tube to form a fixation element. The fixation element may extend between a distal fixation end and a proximal fixation end along a direction of a helical axis. The fixation element may define a proximal fixation section proximate the proximal fixation end, a distal fixation section proximate the distal fixation end, and a middle fixation section located between the proximal and distal fixation sections. Cutting the helical shape may include cutting a smaller cross-sectional dimension for the middle fixation section than a cross-sectional dimension of the proximal fixation section. Further, the method 200 may include cutting 230 a connector from the tube that extends between adjacent portions of the fixation element spaced apart along the helical axis. In other words, the connector may be created or formed at the same time as the fixation element (e.g., the connector is not later mounted or affixed to the fixation element). Although, a manufacturing process of constructing the fixation element (e.g., by cutting a tube structure) and later coupling a connector to the fixation element is contemplated herein. The tube structure may be cut in any suitable way such as, for example, laser cutting, water jet, plasma, etc.

In one or more embodiments, the method may also include cutting at least one opening in the connector. As described herein, the opening may provide some additional flexibility to the connector (and, thus, the fixation element). The at least one opening may define a variety of different shapes including, e.g., a diamond shape, elliptical shape, chevron shape, etc. Further, the method may include cutting any number of openings in the connector. Specifically, the method may include cutting one, two, three, four, etc. opening in the connector. In one or more embodiments, multiple openings may be arranged along the helical axis.

In one or more embodiments, cutting the helical shape from the tube structure may include cutting a smaller cross-sectional dimension for the middle fixation section than a cross-sectional dimension of the distal fixation section. Further, cutting a helical shape from the tube structure may also include forming a taper between the middle fixation section and the distal fixation section and/or forming a taper between the middle fixation section and the proximal fixation section.

In one or more embodiments, the method may also include cutting an additional connector from the tube that extends between adjacent portions of the fixation element spaced apart along the helical axis and spaced apart from the connector.

In one or more embodiments, the method may also include cutting or forming a plurality of features into the fixation element within the distal fixation section (e.g., proximate the distal tip). In other words, the plurality of features may be cut or formed from the tube structure before or after the fixation element is cut from the tube structure (e.g., using the same tools). For example, the method may include cutting a plurality of openings at the distal fixation section of the fixation element (e.g., proximate the distal tip). Also, for example, the method may include cutting a textured surface at the distal fixation section of the fixation element (e.g., proximate the distal tip). Specifically, in one or more embodiments, the openings and/or textured surface may be formed by saw toothing the tube structure.

Alternatively, the fixation element may be cut from a sheet material and formed into a helical structure. For example, FIGS. 8A and 8B illustrate a sheet material 122 including flattened fixation elements 126 (e.g., extending in a plane) arranged along the sheet material 122. The flattened fixation elements 126 may be positioned to maximize the number of fixation elements 126 placed within the sheet material 122. The fixation elements 126 may be cut from sheet material 122 in any suitable way such as, for example, roller cutting, laser cutting, water jet, plasma, etc. Specifically, the fixation elements 126 may be cut from the sheet material 122 in a direction 124 extending along the fixation elements 126 (e.g., as shown in FIG. 8A) or in a direction 124 extending perpendicular to the fixation elements 126 (e.g., as shown in FIG. 8B).

After the flattened fixation elements 126 are cut from the sheet material 122, the fixation elements 126 may be formed into a helical structure (e.g., the fixation element 130) and attached to a body portion of the medical device. Further, in one or more embodiments, a connector may be attached to the fixation element 130 after being formed into a helical structure. In some embodiments, the connector may also be cut from the sheet material 122 (e.g., only attached to the fixation element 126 at one end of the connector). In such embodiments, the connector may be attached to the fixation element 126 after the fixation element 126 is formed into a helical structure (e.g., attaching both ends of the connector or the end of the connector not formed into the fixation element from the sheet material).

Additionally, in one or more embodiments, the cross-sectional shape of the fixation element 130 may be rotated about a centroidal axis. Rotation of the fixation element about the centroidal axis may provide a similar effect as varying the cross-sectional dimension as described herein. In other words, rotation of the fixation element 130 about the centroidal axis may help to optimize performance (e.g., fixation improvement, reduced trauma, longevity or fatigue performance, customizable properties, etc.).

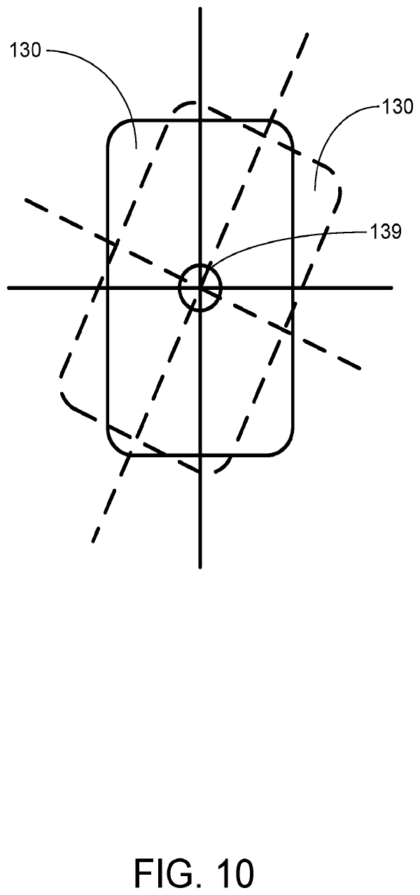

For example, as shown in FIG. 10, the unrotated cross-sectional shape of the fixation element 130 is illustrated in solid lines and defines a centroidal axis 139. The fixation element 130 may be rotated about the centroidal axis 139 to different angles (e.g., as shown in broken lines in FIG. 10). Specifically, the angle of the fixation element 130 (e.g., about the centroidal axis 139) may change between a distal fixation end and a proximal fixation end. The rotation of the fixation element 130 about the centroidal axis 139 may vary from about 0 degrees to 90 degrees. The angle of the fixation element 130 about the centroidal axis 139 may be customizable and vary based on the application.

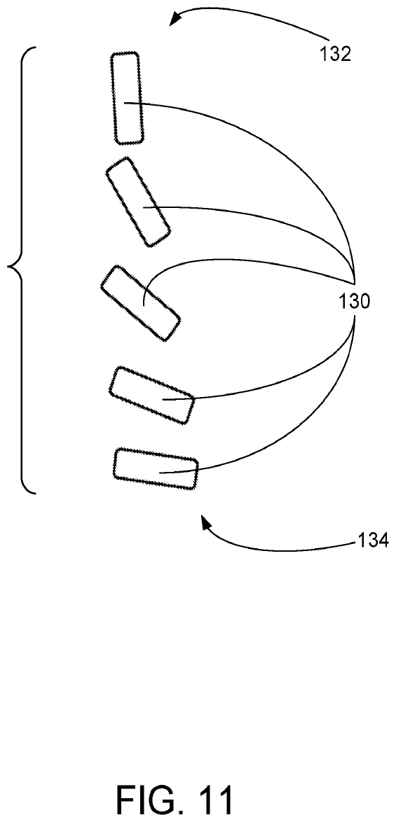

FIG. 11 illustrates a cross-section of multiple subsequent sections of the fixation element 130 from only one side of the helical shape. For example, the fixation element 130 may extend at an angle of about 0 degrees (e.g., relative to a vertical axis) near the distal fixation end 132 and at an angle of about 70 to 90 degrees (e.g., relative to a vertical axis) near the proximal fixation end 134. As such, the angle of the fixation element 130 near the distal fixation end 132 may assist with piercing the tissue and/or the angle of the fixation element 130 near the proximal fixation end 134 may assist with being attached to the body portion of the device.

In one or more embodiments, rotation of the fixation element 130 about the centroidal axis 139 may vary beyond 90 degrees (e.g., at any angle). For example, in one or more embodiments, the fixation element 130 may be twisted about the centroidal axis 139 for one or more rotations (or, e.g., less than one full rotation) as the fixation element 130 is formed into a helical structure. Specifically, the fixation element 130 may define a twisted “ribbon” that defines a rotation rate per unit length that may be (e.g., subsequently) coiled into a helical configuration (e.g., similar to a strand of DNA).

It is noted that the concept of a cross-sectional shape of the fixation element 130 being rotated about a centroidal axis could be in the alternative or in combination with the concept of varying the cross-sectional dimension, as described herein.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, the term “configured to” may be used interchangeably with the terms “adapted to” or “structured to” unless the content of this disclosure clearly dictates otherwise.

The singular forms “a,” “an,” and “the” encompass embodiments having plural referents unless its context clearly dictates otherwise. As used herein, the term “or” refers to an inclusive definition, for example, to mean “and/or” unless its context of usage clearly dictates otherwise. The term “and/or” refers to one or all of the listed elements or a combination of at least two of the listed elements.

As used herein, the phrases “at least one of” and “one or more of” followed by a list of elements refers to one or more of any of the elements listed or any combination of one or more of the elements listed.

As used herein, the terms “coupled” or “connected” refer to at least two elements being attached to each other either directly or indirectly. An indirect coupling may include one or more other elements between the at least two elements being attached. Either term may be modified by “operatively” and “operably,” which may be used interchangeably, to describe that the coupling or connection is configured to allow the components to interact to carry out described or otherwise known functionality. For example, a controller may be operably coupled to a resistive heating element to allow the controller to provide an electrical current to the heating element.

As used herein, any term related to position or orientation, such as “proximal,” “distal,” “end,” “outer,” “inner,” and the like, refers to a relative position and does not limit the absolute orientation of an embodiment unless its context of usage clearly dictates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

### Illustrative Embodiments

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

Thus, various embodiments described herein are disclosed. It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.
A1. An implantable medical device comprising:
a body portion extending between a distal body end and a proximal body end;
a fixation element coupled to the distal body end and extending away from the body portion, wherein the fixation element is configured to affix the body portion to a wall of a heart, wherein the fixation element defines a helical shape extending between a distal fixation end and a proximal fixation end along a direction of a helical axis, wherein the proximal fixation end is coupled to the distal body end,
wherein the fixation element defines a proximal fixation section proximate the proximal fixation end, a distal fixation section proximate the distal fixation end, and a middle fixation section located between the proximal and distal fixation sections, wherein a cross-sectional dimension of the middle fixation section is smaller than a cross-sectional dimension of the proximal fixation section; and
a connector connected between adjacent portions of the fixation element spaced apart along the helical axis, wherein the connector comprises a same material as the fixation element.
A2. The device according to any A embodiment, wherein the cross-sectional dimension of the middle fixation section is smaller than a cross-sectional dimension of the distal fixation section.
A3. The device according to embodiment A2, wherein the fixation element tapers from the middle fixation section to the distal fixation section.
A4. The device according to any A embodiment, wherein the connector defines at least one opening.
A5. The device according to embodiment A4, wherein the at least one opening defines a diamond shape.
A6. The device according to embodiment A4, wherein the at least one opening defines two openings arranged along the helical axis.
A7. The device according to any A embodiment, further comprising an additional connector connected between adjacent portions of the fixation element spaced apart along the helical axis and spaced apart from the connector.
A8. The device according to any A embodiment, wherein the connector defines a width of about 0.3 mm to 0.5 mm.
A9. The device according to any A embodiment, wherein the cross-sectional dimension of the middle fixation section defines a width of about 0.3 mm and the cross-sectional dimension of the proximal fixation section defines a width of about 0.5 mm.
A10. The device according to any A embodiment, wherein the fixation element tapers from the middle fixation section to the proximal fixation section.
B1. A method of manufacturing a fixation element for an implantable medical device, the method comprising:
providing a tube defining a passage therethrough;
cutting a helical shape from the tube to form a fixation element, wherein the fixation element extends between a distal fixation end and a proximal fixation end along a direction of a helical axis, wherein the fixation element defines a proximal fixation section proximate the proximal fixation end, a distal fixation section proximate the distal fixation end, and a middle fixation section located between the proximal and distal fixation sections, wherein cutting the helical shape comprises cutting a smaller cross-sectional dimension for the middle fixation section than a cross-sectional dimension of the proximal fixation section; and
cutting a connector from the tube that extends between adjacent portions of the fixation element spaced apart along the helical axis.
B2. The method according to any B embodiment, wherein the tube defines a thickness of about 0.2 mm to 0.5 mm.
B3. The method according to any B embodiment, further comprising cutting at least one opening in the connector.
B4. The method according to embodiment B3, wherein the at least one opening defines a diamond shape.
B5. The method according to embodiment B3, wherein cutting at least one opening in the connector comprises cutting two openings in the connector, wherein the two openings are arranged along the helical axis.
B6. The method according to any B embodiment, wherein cutting the helical shape comprises cutting a smaller cross-sectional dimension for the middle fixation section than a cross-sectional dimension of the distal fixation section.
B7. The method according to any B embodiment, wherein cutting a helical shape comprises forming a taper between the middle fixation section and the distal fixation section.
B8. The method according to any B embodiment, wherein cutting a helical shape comprises forming a taper between the middle fixation section and the proximal fixation section.
B9. The method according to any B embodiment, further comprising cutting an additional connector from the tube that extends between adjacent portions of the fixation element spaced apart along the helical axis and spaced apart from the connector.
B10. The method according to any B embodiment, wherein the connector defines a width of about 0.3 mm to 0.5 mm.
B11. The method according to any B embodiment, wherein the cross-sectional dimension of the middle fixation section defines a width of about 0.3 mm and the cross-sectional dimension of the proximal fixation section defines a width of about 0.5 mm.
B12. The method according to any B embodiment, further comprising cutting a plurality of openings at the distal fixation section of the fixation element.
B13. The method according to any B embodiment, further comprising cutting a textured surface at the distal fixation section of the fixation element.
C1. An implantable medical device comprising:
a body portion extending between a distal body end and a proximal body end; and
a fixation element coupled to the distal body end and extending away from the body portion, wherein the fixation element is configured to affix the body portion to a wall of a heart, wherein the fixation element defines a helical shape extending between a distal fixation end and a proximal fixation end along a direction of a helical axis, wherein the proximal fixation end is coupled to the distal body end, wherein the fixation element defines a proximal fixation section proximate the proximal fixation end, a distal fixation section proximate the distal fixation end, and a middle fixation section located between the proximal and distal fixation sections, wherein a cross-sectional dimension of the middle fixation section is smaller than a cross-sectional dimension of the proximal fixation section,
wherein fixation element comprises a plurality of features located on a surface of the fixation element proximate the distal fixation end.
C2. The device according to any C embodiment, wherein the plurality of features comprise a plurality of openings extending through the fixation element.
C3. The device according to any C embodiment, wherein the plurality of features comprise a textured surface.
C4. The device according to any C embodiment, wherein the cross-sectional dimension of the middle fixation section is smaller than a cross-sectional dimension of the distal fixation section.
C5. The device according to embodiment C4, wherein the fixation element tapers from the middle fixation section to the distal fixation section.
C6. The device according to any C embodiment, wherein the cross-sectional dimension of the middle fixation section defines a width of 0.3 mm and the cross-sectional dimension of the proximal fixation section defines a width of 0.5 mm.
C7. The device according to any C embodiment, wherein the fixation element tapers from the middle fixation section to the proximal fixation section.
C8. The device of claim 24, further comprising a connector connected between adjacent portions of the fixation element spaced apart along the helical axis, wherein the connector comprises a same material as the fixation element.
C9. The device according to embodiment C8, wherein the connector defines at least one opening.
C10. The device according to embodiment C8, wherein the at least one opening defines a diamond shape.
C11. The device according to embodiment C8, wherein the at least one opening defines two openings arranged along the helical axis.
C12. The device according to embodiment C8, further comprising an additional connector connected between adjacent portions of the fixation element spaced apart along the helical axis and spaced apart from the connector.
C13. The device according to embodiment C8, wherein the connector defines a width of 0.3 mm to 0.5 mm.

1. An implantable medical device comprising:
a body portion extending between a distal body end and a proximal body end;
a fixation element coupled to the distal body end and extending away from the body portion, wherein the fixation element is configured to affix the body portion to a wall of a heart, wherein the fixation element defines a helical shape extending between a distal fixation end and a proximal fixation end along a direction of a helical axis, wherein the proximal fixation end is coupled to the distal body end,
wherein the fixation element defines a proximal fixation section proximate the proximal fixation end, a distal fixation section proximate the distal fixation end, and a middle fixation section located between the proximal and distal fixation sections, wherein a cross-sectional dimension of the middle fixation section is smaller than a cross-sectional dimension of the proximal fixation section; and
a flexible connector connected between adjacent portions of the fixation element spaced apart along the helical axis, wherein the connector comprises a same material as the fixation element.

2. The device of claim 1, wherein the cross-sectional dimension of the middle fixation section is smaller than a cross-sectional dimension of the distal fixation section.

3. The device of claim 2, wherein the fixation element tapers from the middle fixation section to the distal fixation section.

4. The device of claim 1, wherein the flexible connector defines at least one opening.

5. The device of claim 1, wherein the flexible connector defines a width of 0.3 mm to 0.5 mm.

6. The device of claim 1, wherein the fixation element tapers from the middle fixation section to the proximal fixation section.

7. A method of manufacturing a fixation element for an implantable medical device, the method comprising:
providing a tube defining a passage therethrough;
cutting a helical shape from the tube to form a fixation element, wherein the fixation element extends between a distal fixation end and a proximal fixation end along a direction of a helical axis, wherein the fixation element defines a proximal fixation section proximate the proximal fixation end, a distal fixation section proximate the distal fixation end, and a middle fixation section located between the proximal and distal fixation sections, wherein cutting the helical shape comprises cutting a smaller cross-sectional dimension for the middle fixation section than a cross-sectional dimension of the proximal fixation section; and
cutting a flexible connector from the tube that extends between adjacent portions of the fixation element spaced apart along the helical axis.

8. The method of claim 7, wherein the tube defines a thickness of 0.2 mm to 0.5 mm.

9. The method of claim 7, further comprising cutting at least one opening in the flexible connector.

10. The method of claim 7, wherein cutting the helical shape comprises cutting a smaller cross-sectional dimension for the middle fixation section than a cross-sectional dimension of the distal fixation section.

11. The method of claim 7, wherein the flexible connector defines a width of 0.3 mm to 0.5 mm.

12. The method of claim 7, further comprising cutting a plurality of openings at the distal fixation section of the fixation element.

13. The method of claim 7, further comprising cutting a textured surface at the distal fixation section of the fixation element.

14. An implantable medical device comprising:
a body portion extending between a distal body end and a proximal body end; and
a fixation element coupled to the distal body end and extending away from the body portion, wherein the fixation element is configured to affix the body portion to a wall of a heart, wherein the fixation element defines a helical shape extending between a distal fixation end and a proximal fixation end along a direction of a helical axis, wherein the proximal fixation end is coupled to the distal body end,
wherein the fixation element defines a proximal fixation section proximate the proximal fixation end, a distal fixation section proximate the distal fixation end, and a middle fixation section located between the proximal and distal fixation sections, wherein a cross-sectional dimension of the middle fixation section is smaller than a cross-sectional dimension of the proximal fixation section,
wherein fixation element comprises a plurality of features located on a surface of the fixation element proximate the distal fixation end.

15. The device of claim 14, wherein the plurality of features comprise a plurality of openings extending through the fixation element.

16. The device of claim 14, wherein the plurality of features comprise a textured surface.

17. The device of claim 14, wherein the cross-sectional dimension of the middle fixation section is smaller than a cross-sectional dimension of the distal fixation section.

18. The device of claim 14, further comprising a connector connected between adjacent portions of the fixation element spaced apart along the helical axis, wherein the connector comprises a same material as the fixation element.

19. The device of claim 18, wherein the connector defines at least one opening.

20. The device of claim 18, wherein the connector defines a width of 0.3 mm to 0.5 mm.
